(19) United States
(12) Patent Application Publication
(10) Pub. No.: US 2022/0054191 A1
(43) Pub. Date: Feb. 24, 2022

(54) DEVICE FOR COLLAGENOUS TISSUE TREATMENT

(71) Applicant: Inmode Ltd., Yokneam (IL)

(72) Inventor: Michael Kreindel, Richmond Hill (CA)

(73) Assignee: Inmode Ltd., Yokneam (IL)

(21) Appl. No.: 17/518,806

(22) Filed: Nov. 4, 2021

Related U.S. Application Data
(63) Continuation of application No. 15/981,039, filed on May 16, 2018, now abandoned.

(51) Int. Cl. A61B 18/14; A61B 18/16

(52) U.S. Cl. CPC A61B 18/1485; A61B 2018/00559; A61B 18/16

(57) ABSTRACT

A method of contraction of collagenous tissue between urethra and vagina includes positioning an RF active electrode between a urethra and a vagina and delivering RF energy into the collagenous tissue. The method includes using a mechanism to direct the active electrode into the safe position to avoid damage of important structures.

### CROSS-REFERENCE TO OTHER APPLICATIONS

This application claims priority from, and is a continuation of, U.S. patent application Ser. No. 15/981039, filed 16 May 2018, which is incorporated herein by reference.

### FIELD OF THE INVENTION

The invention relates to methods for minimally invasive treatment of severe urinary incontinence using RF energy and safe positioning of an RF electrode.

### BACKGROUND OF THE INVENTION

There are many surgical and non-surgical methods for treatment severe urinary incontinence (SUI) using RF energy.

For example, in U.S. Pat. No. 7,792,589, there is a method that includes remodeling of collagenous tissue by applying RF energy with a hand piece inserted into the vagina and creating heat to contract the collagenous support tissue.

U.S. Pat. 6,463,331 describes a device for SUI treatment comprising multiple electrodes adapted to deliver RF energy to urethral tissue.

U.S. Pat. 7,315,762 describes a method for treating incontinence by heating collagenous pelvic tissue with a probe.

U.S. Pat. Nos. 6,216,704, 6,139,569 and 6,480,746 describe shrinking a target collagenous endopelvic support tissue with RF energy.

U.S. Pat. No. 6,836,688 describes treatment of the urethra, using thermally induced shrinkage of the pelvic floor.

All prior art methods of treatment contract tissue that supports the urethra in an effort to reduce urine incontinence. RF energy is used to induce thermal contraction of the tissue and this energy is delivered invasively or non-invasively to create tissue shrinkage without damage to the vaginal wall or urethra.

### SUMMARY OF THE INVENTION

The present invention describes a method of positioning an active RF electrode in the tissue between the urethra and the vaginal wall for safe and effective contraction of collagenous tissue supporting the urethra.

The active electrode is positioned at a safe distance between the urethra and the vagina to deliver a therapeutic amount of energy and create shrinkage of supporting tissue. The device is based on a minimally invasive procedure in which at least one active electrode is inserted in soft tissue enclosed to the urethra. The size of the electrode is designed to create higher energy density in the vicinity of the electrode. The RF energy density should be high enough to create contraction of collagenous tissue. The method uses a system which has a rigid position fixator designed to be inserted into the urethra and a directing element for inserting an active electrode substantially parallel to the urethra into the tissue at a predetermined distance from the urethra canal.

In one embodiment, the distance between the urethral position fixator and the active electrode is fixed. Alternatively, the distance between the active electrode and the urethral position fixator can be adjusted by the user according to the individual anatomy of the patient.

The position fixator may have smooth surfaces and a blunt end to avoid damage to the urethra. Lubrication can be used for easy insertion.

The internal electrode may be inserted into the tissue to a depth of 3-40 mm to provide enough collagen contraction to support the urethra. The active electrode is sufficiently rigid to avoid undesired bending in the tissue.

The active electrode may have an insulated shaft and a conductive area at the distal end of the electrode. The electrode may have a non-conductive blunt tip to avoid electro-surgical and mechanical perforation of the important internal organs.

The active electrode may have an embedded temperature sensor to control tissue heating up to the temperature providing collagen contraction. Typical, heating temperature is varied from 45° C. up to 100° C.

During the treatment the electrode is inserted into the distal position and RF energy is applied during retraction to create collagen contraction around the electrode

In one embodiment the active electrode is connected to the RF generator by a conductor passing through the dielectric lumen.

The external electrode can be applied to the external body area utilizing a mono-polar design of the system.

Alternatively, the return electrode can be connected to the active electrode and inserted into the vagina and stay close to the active electrode.

The return electrode may have a substantially larger conductive area than the active electrode to avoid tissue thermal damage in the area coupled to the external electrode. The return electrode can be structured from one or more conductive elements. The return electrode may have an embedded thermal sensor to avoid tissue overheating.

The active and return electrodes can be connected mechanically to control the distance between electrodes. The connection between them may have different predetermined positions in order to fix specific distances between the electrodes, and to control the treatment depth. The connection between the electrodes may have a biasing device (e.g., a spring) to compress tissue between the electrodes.

The part of the electrodes that contacts the tissue may be made of biocompatible materials. For example, the internal electrode tip can be made of stainless steel or titanium. RF electrodes may have a thin dielectric coating providing capacitive electrical coupling for delivering RF energy.

The parameters of the RF energy may be adjusted for tissue contraction. RF energy can be delivered in pulsed or continuous mode. Frequency of the RF current may vary from 200 KHz up to 40 MHz. Generation of higher frequency is more complicated technically but provides better coupling with tissue. In order to improve electrical coupling, a conductive solution can be applied to the return electrode. Conductive liquid or gel can be used to hydrate tissue under the return electrode and to improve electrical contact. RF energy can be controlled by controlling the RF power. Another option to control average RF power is to deliver constant RF power with a train of pulses and control the duty cycle of the RF pulses.

In order to reduce pain and improve tissue conductivity, tumescent anesthesia can be applied to the treated tissue prior the procedure.

Controlling the distance between the active electrode, the urethral fixator and the return electrode placed in the vagina allows optimizing safe positioning of the RF electrode between the urethra and the vagina.

In some embodiments, the device may have circuit for measuring tissue impedance. Change of measured impedance between electrodes may provide information about the distance between electrodes. Measuring of the tissue impedance also provides information about tissue heating and quality of electrical contact between the return electrode and the tissue surface. The electronic circuit may measure RF current, voltage, impedance or other parameters.

The tip of the active electrode may be located above the return electrode. This may prevent delivering of excessive energy to the tissue.

Cooling of the return electrode and/or the urethral fixator may reduce risk of side effects.

The system for powering and controlling RF energy delivery may include a power supply that converts AC voltage to a stabilized DC voltage. An RF generator may be connected to the power supply for generating high frequency voltage. The RF generator may be designed to maintain constant power in the working range of parameters. The system may have a controller that controls the RF parameters and a user interface that includes an LCD screen and touch panel. The controller may have a microprocessor and dedicated software. The monitoring system is required to measure RF parameters including, but not limited to, tissue impedance, RF current, RF voltage or other electronic parameters. The system may have a connector to connect one or more electrodes to the system unit.

Thus a method is provided for contracting supporting tissue between urethra and vagina by:

inserting a position fixator into a urethra;

aligning an active electrode through a directing element into tissue between the urethra and vagina;

applying a return electrode into a wall of the vagina under a conductive area of the active electrode;

applying RF energy to heat tissue in a vicinity of the conductive area of the active electrode;

monitoring temperature of tissue around the active electrode and temperature under the return electrode;

maintaining temperature of tissue around the active electrode at a predetermined therapeutic level by adjusting delivered RF power;

stopping RF energy delivery after a predetermined treatment time or when a safety temperature threshold under the return electrode is reached; and

repositioning the active and return electrodes to a new position to treat another area of tissue.

A method is provided for contracting supporting tissue between urethra and vagina by:

inserting a position fixator into a urethra;

aligning an active electrode through a directing element into tissue between the urethra and vagina;

applying a return electrode into a wall of the vagina under a conductive area of the active electrode;

applying RF energy to heat tissue in a vicinity of a conductive area of the active electrode;

moving the active electrode back and forth over a treated tissue volume;

monitoring temperature of tissue around the active electrode and temperature under the return electrode;

maintaining temperature of tissue around the active electrode at a predetermined therapeutic level by adjusting delivered RF power;

stopping RF energy delivery after a predetermined treatment time or when a required amount of energy is delivered to the treatment volume.

A method is provided for contracting supporting tissue between urethra and vagina by:

inserting a position fixator into a urethra;

aligning an active electrode through a directing element into tissue at a predetermined distance from the urethra;

applying a return electrode into a patient body;

applying RF energy to heat tissue in a vicinity of a conductive area of the active electrode;

monitoring temperature of tissue around the active electrode;

maintaining temperature of tissue around the active electrode at a predetermined therapeutic level by adjusting delivered RF power;

stopping RF energy delivery after a predetermined treatment time or after a predetermined amount of RF energy is delivered; and

repositioning the active electrode to a new position to treat another tissue volume.

### BRIEF DESCRIPTION OF THE DRAWINGS

In the accompanying drawings:

FIG. 1 illustrates a female urinary and reproductive system;

FIG. 2 illustrates an electro-surgical instrument with active and return electrodes, in accordance with an embodiment of the invention;

FIG. 3 illustrates a directing mechanism for aligning an internal electrode into the tissue, in accordance with an embodiment of the invention; and

FIG. 4 illustrates the electro-surgical instrument and directing mechanism applied to the patient's body.

### DETAILED DESCRIPTION OF THE INVENTION

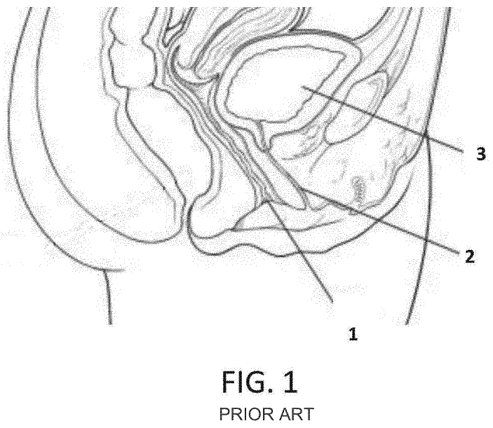

FIG. 1 illustrates anatomy of a female urinary and reproductive system. Urethra 2 is connected to the urinary bladder 3. The urethra 2 is located above the vagina 1 and is virtually parallel to the vagina 1. In order to achieve a desired therapeutic effect, the supporting tissue between urethra 2 and vagina 1 may be made firmer by contraction of collagen. The treatment may be performed in this delicate area to avoid damage to urethra 2, bladder 3 and vagina 1.

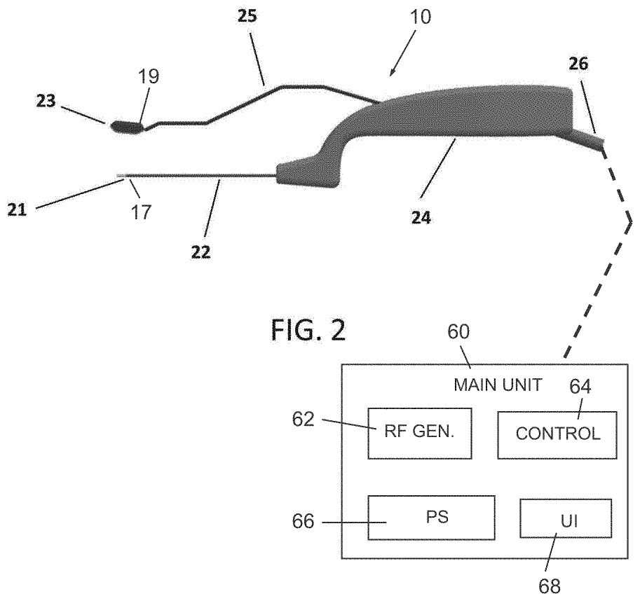

FIG. 2 illustrates an electro-surgical instrument 10 including a handle 24, an active electrode 22 and a return electrode 23. The shaft of active electrode 22 is coated with an insulating material and has a conductive tip 21 to deliver RF energy at the distal end of the electrode 22. The insulating material is thick enough to prevent significant RF leakage through capacitive coupling. Conductive tip 21 may have a length of 1 mm to 10 mm, without limitation. The diameter of active electrode 22 may be, without limitation, in a range of 0.7-2.5 mm to prevent physical damage to the patient during treatment. Return electrode 23 has a substantially larger area than the conductive area 21 of active electrode 22. Return electrode 23 may be connected to the handle 24 by means of a biasing device, such as a spring arm 25. The user may adjust the distance between the tip of active electrode 22 and return electrode 23 by adjusting spring arm 25. A thermistor or other temperature sensor 17 may be disposed on or in a tip of active electrode 22 to measure the temperature of tissue during treatment. Another thermistor or other temperature sensor 19 may be disposed on or in the return electrode 23 to monitor temperature of tissue coupled to the return electrode 23. RF energy and signals from both thermistors 17 and 19 may be delivered through a harness 26 to a main unit 60. The main unit 60 includes an RF generator 62, controller unit 64, power supply 66 and user interface 68. The main unit 60 delivers RF energy to the hand piece 24 according to user settings, RF impedance measurements and feedback obtained from both thermistors 17 and 19.

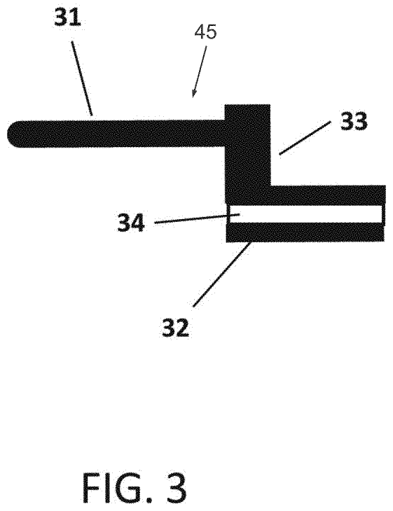

FIG. 3 illustrates a directing mechanism 45 that controls positioning of active electrode 22 between the urethra and vagina. The mechanism 45 includes a urethral positioning fixator 31 which can be inserted into the urethra. The fixator 31 may be, without limitation, a cylindrical pin with a diameter suitable for insertion into the urethra and a length longer than its diameter. A guide 32 is coupled to fixator 31 by a connecting element 33. Guide 32 has a lumen 34 for the active electrode 22 to pass through. Lumen 34 may be parallel to fixator 31. Active electrode 22 may be placed at any desired position along lumen 34 so that it is placed at a desired depth parallel to the urethra. The directing mechanism 45 avoids accidental damage to the urethra by active electrode 22.

The connecting element 33 may be telescopic, and may be pivotable and lockable at any desired orientation with respect to fixator 31 and/or guide 32, so as to accommodate different anatomical geometries the user may encounter.

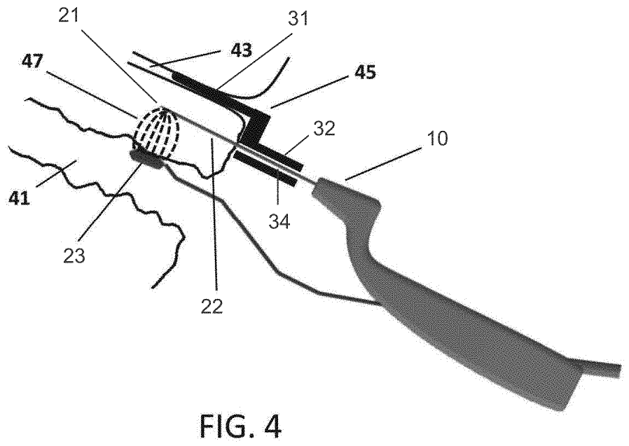

FIG. 4 schematically illustrates a method of treatment using electro-surgical instrument 10 with directing mechanism 45. In one non-limiting embodiment, the method of treatment includes the following steps:

Inserting the fixator 31 of directing mechanism 45 into a urethra 43.

Positioning the lumen 34 of the directing mechanism 45 in front of (anterior to) the desired treatment volume between urethra 43 and vagina 41. The connecting element 33 may be adjusted to position lumen 34 at the correct place.

Inserting active electrode 22 into the tissue through the lumen 34 of directing mechanism 45 and applying return electrode 23 to the wall of the vagina 41.

Applying RF energy 47 between conductive tip 21 of active electrode 22 and return electrode 23 to create collagen contraction in vicinity of active electrode 22. Using the method of the invention to treat urethra supporting tissue, the following non-limiting parameter values of RF energy may be used:

RF frequency: 0.2-40 MHz.

Average output RF power: from about 0.5 to about 300 W.

Delivered energy creates a sufficiently high temperature to contract collagen in the vicinity of active electrode conductive tip.

1. An apparatus for tissue treatment comprising:
a directing mechanism comprising a guide formed with a lumen that extends through a longitudinal axis of said guide, said lumen being open at a distal end thereof;
a connecting element coupled to said guide;
a urethral positioning fixator coupled to said connecting element, a longitudinal axis of said urethral positioning fixator being offset from the longitudinal axis of said lumen of said guide, said ;
an active electrode arranged to pass through said lumen and protrude past said distal end of said lumen;
a return electrode offset from said longitudinal axis of said lumen of said guide such that said urethral positioning fixator and said return electrode are located on opposite sides of said guide; and
a main unit comprising an RF generator, a controller unit, a power supply and a user interface, said main unit configured to deliver RF energy between said return electrode and a conductive tip of said active electrode to create collagen contraction in a vicinity of said active electrode.

2. The apparatus according to claim 1, wherein said connecting element has an adjustable length that changes an amount said longitudinal axis of said urethral positioning fixator is offset from said longitudinal axis of said lumen of said guide.

3. The apparatus according to claim 1, wherein said connecting element is pivotable and lockable at any desired orientation with respect to said urethral positioning fixator or said guide.

4. The apparatus according to claim 1, wherein said urethral positioning fixator is parallel to said lumen of said guide.

5. The apparatus according to claim 1, wherein said urethral positioning fixator is parallel to said active electrode.

6. The apparatus according to claim 1, wherein said connecting element is perpendicular to said urethral positioning fixator.

7. The apparatus according to claim 1, wherein said connecting element is perpendicular to said guide.

8. A method for tissue treatment comprising using the apparatus of claim 1 and:
inserting said urethral positioning fixator into a urethra of a patient;
passing said active electrode through said distal end of said lumen so that said active RF electrode passes into tissue between the urethra and a vagina of the patient;
inserting a return electrode into the vagina; and
applying RF energy between said active electrode and said return electrode to create collagen contraction in a vicinity of said active electrode to contract collagenous tissue between the urethra and the vagina.

9. The method according to claim 8, wherein said active RF electrode has a distal conductive tip and an insulating shaft and the RF energy is delivered to said conductive tip.

10. The method according to claim 8, comprising using the RF energy to heat the tissue between the urethra and the vagina above 45° C.

11. The method according to claim 8, further comprising measuring temperature at said active electrode or said return electrode and using the temperature to control RF energy delivery to said active electrode.

12. The method according to claim 8, wherein the return electrode has a substantially larger area than a conductive area of the active electrode.

13. The method according to claim 8, further comprising adjusting a length of said connecting element so as to change an amount said longitudinal axis of said urethral positioning fixator is offset from said longitudinal axis of said lumen of said guide.
